(19) United States
(12) Patent Application Publication
(10) Pub. No.: US 2023/0127147 A1
(43) Pub. Date: Apr. 27, 2023

(54) CONTINUOUS-FLOW PREPARATION METHOD OF DIOL SULFONE

(71) Applicant: Fudan University, Shanghai (CN)

(72) Inventors: Fener CHEN, Shanghai (CN); Minjie LIU, Shanghai (CN); Meifen JIANG, Shanghai (CN); Yingqi XIA, Shanghai (CN); Huashan HUANG, Shanghai (CN)

(21) Appl. No.: 18/068,151

(22) Filed: Dec. 19, 2022

(30) Foreign Application Priority Data
Mar. 28, 2022 (CN) 202210313702.X

(51) Int. Cl. C07C 315/02; C07C 315/06

(52) U.S. Cl. CPC C07C 315/02; C07C 315/06

(57) ABSTRACT

A continuous-flow preparation method of diol sulfone using a two-stage micro-reaction system. The system includes a first micro-mixer, a first micro-channel reactor, a second micro-mixer, and a second micro-channel reactor communicated in sequence. The method includes: feeding hydrogen peroxide, a catalyst and a diol thioether solution simultaneously to the first micro-mixer followed by mixing; feeding the reaction mixture to the first micro-channel reactor for continuous oxidation; and feeding the reaction mixture and water simultaneously to the second micro-mixer and the second micro-channel reactor for continuous quenching and crystallization to obtain diol sulfone.

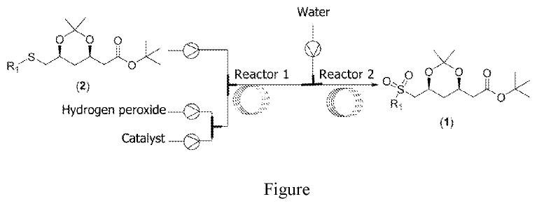

### CROSS-REFERENCE TO RELATED APPLICATIONS

This application claims the benefit of priority from Chinese Patent Application No. 202210313702.X, filed on Mar. 28, 2022. The content of the aforementioned application, including any intervening amendments thereto, is incorporated herein by reference.

### TECHNICAL FIELD

This application relates to organic chemical engineering, and more specifically to a continuous-flow preparation method of diol sulfone.

### BACKGROUND

Diol sulfone (compound 1) is an important pharmaceutical and chemical intermediate for the synthesis of hypolipidemic medicines such as rosuvastatin calcium, atorvastatin calcium and pitavastatin calcium, and has a wide application prospect in the pharmaceutical industry.

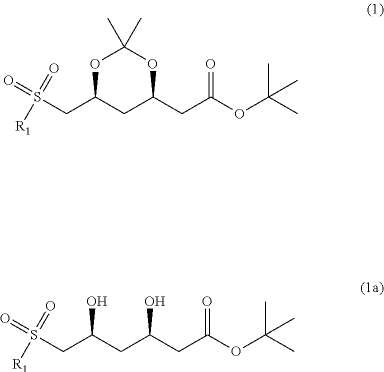

U.S. Patent Publication No. 20130303779A1 discloses a method of preparing the compound (1) using expensive m-chloroperoxybenzoic acid as the oxidant. This method is time-consuming and has a poor yield (merely 59%). Chinese Patent Publication No. 102459196A and International Patent Application Publication No. 2016125086A1 disclose another preparation method using hydrogen peroxide as the oxidant. This method is also time-consuming and has a poor yield (about 50-55%) due to the generation of about 10% of the diol de-protected product (compound 1a).

Besides the intrinsic defects, the above methods further struggle with high safety risk due to the use of a traditional batch reactor, low efficiency, and high energy consumption, and thus not suitable for the industrial production.

### SUMMARY

To overcome the defects of time-consuming reaction process, generation of various side products, high energy consumption, and low efficiency in the existing synthesis approaches using a traditional batch reactor, this application provides a continuous-flow preparation method of diol sulfone. The method provided herein is characterized by shortened reaction time, less generation of side products, high yield, enhanced automation degree and efficiency, and good safety, and is thus suitable for industrial application.

Technical solutions of this application are described as follows.

This application provides a continuous-flow preparation method of diol sulfone using a two-stage micro-reaction system, the two-stage micro-reaction system comprising a first micro-mixer, a first micro-channel reactor, a second micro-mixer, and a second micro-channel reactor communicated in sequence; and the method comprising:

(S1) simultaneously feeding a hydrogen peroxide solution, a catalyst, and a solution of diol thioether (2) to the first micro-mixer followed by mixing to obtain a reaction mixture; and feeding the reaction mixture to the first micro-channel reactor to undergo a continuous oxidation reaction; and

(S2) feeding the reaction mixture flowing out from the first micro-channel reactor and water to the second micro-mixer for mixing followed by continuous quenching and crystallization in the second micro-channel reactor to obtain diol sulfone (1); as shown in the following reaction scheme:

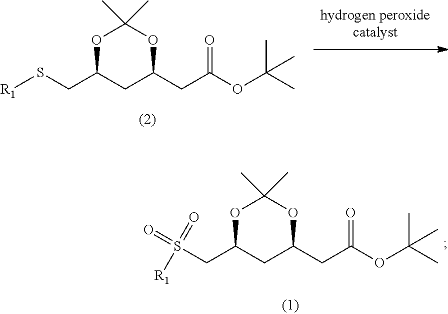

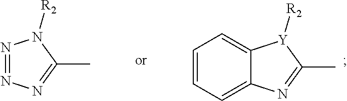

wherein R₁ is Y is nitrogen atom or sulfur atom; and R₂ is a C₁-C₆ alkyl group or phenyl group.

In an embodiment, R1 is methyl tetrazole, tert-butyl tetrazole, or benzothiazole.

In an embodiment, in step (S1), the solution of diol thioether is a solution of diol thioether in an organic solvent; the organic solvent is selected from the group consisting of an alcohol, an ester, and a halogenated hydrocarbon; the alcohol is a C₁-C₆ monohydric alkyl alcohol or a C₁-C₆ polyhydric alkyl alcohol; the ester is selected from the group consisting of methyl acetate, ethyl acetate, and tert-butyl acetate; and the halogenated hydrocarbon is selected from the group consisting of dichloromethane, chloroform, carbon tetrachloride, dichloroethane, and chlorobenzene.

In an embodiment, the organic solvent is methanol or ethanol.

In an embodiment, in step (S1), the hydrogen peroxide solution comprises 10-50% by weight of hydrogen peroxide.

In an embodiment, in step (S1), the hydrogen peroxide solution comprises 30% by weight of hydrogen peroxide.

In an embodiment, the catalyst is a metal catalyst or a non-metal catalyst; and the metal catalyst is selected from the group consisting of a tungsten catalyst, a molybdenum catalyst, a vanadium catalyst, a titanium catalyst, and an iron catalyst.

In an embodiment, the catalyst is ammonium molybdate tetrahydrate or sodium tungstate dihydrate.

In an embodiment, in step (S1), a flow ratio of the solution of diol thioether to the hydrogen peroxide solution is controlled such that a molar ratio of the diol thioether to hydrogen peroxide is 1:(2-10), preferably, 1:(3-5).

In an embodiment, in step (S1), the first micro-channel reactor is controlled at 0-100° C., preferably, 50-70° C.; and a residence time of the reaction mixture in the first micro-channel reactor is controlled to be 0.1-30 min, preferably, 5-8 min.

In an embodiment, in step (S2), a flow ratio of the reaction mixture to the water is controlled such that a molar ratio of the organic solvent to the water is 1:(0.5-5), preferably, 1:(1-2).

In an embodiment, in step (S2), the second micro-channel reactor is controlled at −10-30° C., preferably, 0-10° C.; and a residence time of the reaction mixture in the second micro-channel reactor is controlled to be 0.5-10 min, preferably, 3-5 min.

In an embodiment, the first micro-mixer and the second micro-mixer are independently selected from the group consisting of a static mixer, a T-type micro-mixer, a Y-type micro-mixer, a cross-type micro-mixer, a coaxial-flow micro-mixer, and a flow-focusing micro-mixer, preferably, the group consisting of the cross-type micro-mixer, the coaxial-flow micro-mixer, and the flow-focusing micro-mixer.

In an embodiment, the first micro-channel reactor and the second micro-channel reactor are independently a tubular micro-channel reactor, a plate-type micro-channel reactor, or other types of micro-channel reactors available on the market, preferably, a tubular micro-channel reactor.

In an embodiment, an inner diameter of the tubular micro-channel reactor is 50 μm-10 mm, and preferably, 100 μm-5 mm; the plate-type micro-channel reactor comprises a first heat exchange layer, a reaction layer, and a second heat exchange layer successively arranged from top to bottom; the reaction layer is provided with a reaction fluid channel; and a hydraulic diameter of the reaction fluid channel is 50 μm-10 mm, preferably, 100 μm-5 mm.

In the continuous-flow preparation method of diol sulfone (1), the micro-reaction system is adopted, which can realize the continuous-flow industrial mass production through the strategy of multi-channel parallel amplification.

Compared with the prior art, this application has the following beneficial effects.

The preparation method provided herein employs a micro-reaction system including a micro-mixer and a micro-channel reactor successively communicated. Compared with the existing synthetic method using a conventional batch reactor, the method provided herein has the following advantages.

(1) The method provided herein effectively inhibits the production of side products and increases the yield of the target product.

(2) The continuous flow micro-channel reaction system used herein has excellent performances of mass exchange, heat exchange and material molecular mixing, which greatly reduces the reaction time and greatly improves the reaction efficiency.

(3) The method provided herein realizes the continuous synthesis from raw materials to the target products, has a high degree of automation, no external intervention, and a high space-time efficiency, significantly reducing labor force and intensity and lowering the production cost.

(4) The micro-channel reactor adopted herein can industrially amplify the method provided herein through the strategy of multi-channel parallel amplification to achieve the mass production.

### BRIEF DESCRIPTION OF THE DRAWINGS

This FIGURE schematically illustrates a method for preparing diol sulfone according to an embodiment of the present disclosure.

### DETAILED DESCRIPTION OF EMBODIMENTS

To describe the contents, features, objects and effects of the technical solutions in detail, this application will be described below with reference to the embodiments and drawing. It should be noted that the embodiments are merely illustrative to facilitate the understanding and implementation of the present disclosure, and are not intended to limit the present disclosure.

A micro-reaction system used herein is structurally depicted in the FIGURE, and operated as follows.

(A) An organic solution of compound (2), hydrogen peroxide and a catalyst solution are simultaneously fed into a first micro-channel reactor through a feeding pump to undergo continuous oxidation reaction to obtain a reaction mixture.

(B) The reaction mixture flowing out from the first micro-channel reactor and water are simultaneously fed into the second micro-channel reactor to undergo continuous quenching, crystallization, and filtration to obtain diol sulfone.

### Example 1

Provided herein was a method of continuously preparing tert-butyl 2-((4R,6S)-6-((benzothiazol-2-ylsulfonyl)methyl)-2,2-dimethyl-1,3-dioxan-4-yl) acetate.

A solution of tert-butyl 2-((4R,6S)-6-((benzothiazol-2-ylthio)methyl)-2,2-dimethyl-1,3-dioxan-4-yl) acetate (2) in isopropanol, a 30% (by weight) hydrogen peroxide solution, and an aqueous ammonium molybdate tetrahydrate solution were simultaneously fed into a T-type mixer for mixing, and then the reaction mixture was fed to a first tubular micro-channel reactor (with a reaction volume of 30 mL, and a diameter of 2 mm) and reacted at 70° C. for 8 minutes (namely, a residence time of the reaction mixture in the first tubular micro-channel reactor was 8 minutes), where flow rates of individual raw materials were adjusted such that a molar ratio of the compound (2) to hydrogen peroxide to the ammonium molybdate tetrahydrate was 1:3:0.05, and a back pressure of a back pressure valve was set to be 0.2 MPa. Then the reaction mixture flowing out from the outlet of the first tubular micro-channel reactor (the reaction mixture was sampled and detected, and the conversion rate of the compound (2) was 100%) and water were simultaneously fed into a Y-type micro-mixer for mixing, and then the reaction mixture was fed to a second tubular micro-channel reactor (with a reaction volume of 20 mL, and a diameter of 2 mm) and subjected to quenching and crystallization at 20° C. for 3 minutes (namely, a residence time of the second tubular micro-channel reactor was 3 minutes), where flow rates of the reaction mixture and the water were adjusted such that a volume ratio of isopropanol to the water was 1:1. The second micro-channel reactor was controlled at. The residence time of the reaction mixture in the second micro-channel reactor was. After that, the reaction mixture was allowed to flow out from an outlet of the second micro-channel reactor and filtered to obtain tert-butyl 2-((4R,6S)-6-((benzothiazol-2-ylsulfonyl)methyl)-2,2-dimethyl-1,3-dioxan-4-yl) acetate (1) which had a yield of 80% and a purity of 99% (HPLC).

### Example 2

The preparation method provided in Example 2 was basically the same as that in Example 1 except that in this example, the compound (2) was tert-butyl 2-((4R,6S)-6-((1-methyl-1H-tetrazol-5-ylthio)methyl)-2,2-dimethyl-1,3-dioxan-4-yl) acetate. In this example, the substrate experienced a complete conversion, and the target product had a yield of 78% and a purity of 99% (HPLC).

### Example 3

The preparation method provided in Example 3 was basically the same as that in Example 1 except that in this example, the compound (2) was tert-butyl 2-((4R,6S)-6-((1-tert-butyl-1H-tetrazol-5-ylthio)methyl)-2,2-dimethyl-1,3-dioxan-4-yl) acetate. In this example, the substrate experienced a complete conversion, and the target product had a yield of 83% and a purity of 99% (HPLC).

### Example 4

The preparation method provided in Example 4 was basically the same as that in Example 1 except that in this example, the compound (2) was tert-butyl 2-((4R,6S)-6-((1-phenyl-1H-tetrazol-5-ylthio)methyl)-2,2-dimethyl-1,3-dioxan-4-yl) acetate. In this example, the substrate experienced a complete conversion, and the target product had a yield of 77% and a purity of 98% (HPLC).

### Example 5

The preparation method provided in Example 5 was basically the same as that in Example 1 except that in this example, the solvent was ethanol. In this example, the substrate experienced a complete conversion, and the target product had a yield of 75% and a purity of 98% (HPLC).

### Example 6

The preparation method provided in Example 6 was basically the same as that in Example 1 except that in this example, the catalyst was sodium tungstate dihydrate. In this example, the substrate experienced a complete conversion, and the target product had a yield of 78% and a purity of 99% (HPLC).

### Example 7

The preparation method provided in Example 7 was basically the same as that in Example 1 except that in this example, the oxidation in the first tubular micro-channel reactor was performed at 60° C. In this example, the substrate experienced a complete conversion, and the target product had a yield of 82% and a purity of 99% (HPLC).

### Example 8

The preparation method provided in Example 8 was basically the same as that in Example 1 except that in this example, the oxidation in the first tubular micro-channel reactor was performed at 90° C. In this example, the substrate experienced a complete conversion, and the target product had a yield of 78% and a purity of 98% (HPLC).

### Example 9

The preparation method provided in Example 9 was basically the same as that in Example 1 except that in this example, the oxidation was performed under temperature gradient control. Specifically, the oxidation reaction was performed at 90° C. for 2 min with a reaction volume of 10 mL and a microchannel diameter of 2 mm, and then at 70° C. for 4 min with a reaction volume of 20 mL and a microchannel diameter of 4 mm. In this example, the substrate experienced a complete conversion, and the target product had a yield of 80% and a purity of 98% (HPLC).

It should be noted that the above examples are only used to illustrate the technical solutions of the disclosure, and are not intended to limit the disclosure. It should be understood that any changes, replacements and modifications made by those skilled in the art without departing from the spirit of the disclosure shall fall within the scope of the present disclosure defined by the appended claims.

What is claimed is:

1. A continuous-flow preparation method of diol sulfone using a two-stage micro-reaction system, the two-stage micro-reaction system comprising a first micro-mixer, a first micro-channel reactor, a second micro-mixer, and a second micro-channel reactor communicated in sequence; and the method comprising:
(S1) simultaneously feeding a hydrogen peroxide solution, a catalyst, and a solution of diol thioether (2) to the first micro-mixer followed by mixing to obtain a reaction mixture; and feeding the reaction mixture to the first micro-channel reactor to undergo a continuous oxidation reaction; and
(S2) feeding the reaction mixture flowing out from the first micro-channel reactor and water to the second micro-mixer for mixing followed by continuous quenching and crystallization in the second micro-channel reactor to obtain diol sulfone (1); as shown in the following reaction scheme: 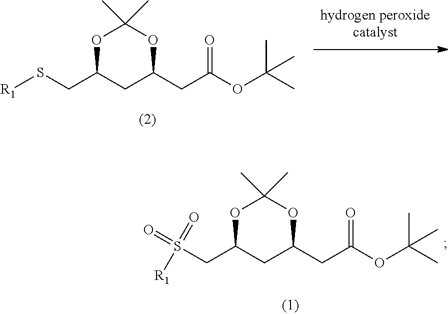
wherein R₁ is 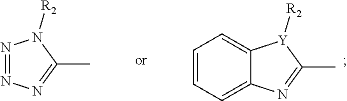
Y is nitrogen or sulfur; and R₂ is a C₁-C₆ alkyl or phenyl.

2. The continuous-flow preparation method of claim 1, wherein in step (S1), the solution of diol thioether is a solution of diol thioether in an organic solvent; the organic solvent is selected from the group consisting of an alcohol, an ester, and a halogenated hydrocarbon; the alcohol is a C₁-C₆ monohydric alkyl alcohol or a C₁-C₆ polyhydric alkyl alcohol; the ester is selected from the group consisting of methyl acetate, ethyl acetate, and tert-butyl acetate; and the halogenated hydrocarbon is selected from the group consisting of dichloromethane, chloroform, carbon tetrachloride, dichloroethane, and chlorobenzene;
the hydrogen peroxide solution comprises 10-50% by weight of hydrogen peroxide; and
the catalyst is a metal catalyst or a non-metal catalyst; and the metal catalyst is selected from the group consisting of a tungsten catalyst, a molybdenum catalyst, a vanadium catalyst, a titanium catalyst, and an iron catalyst.

3. The continuous-flow preparation method of claim 1, wherein in step (S1), a flow ratio of the solution of diol thioether to the hydrogen peroxide solution is controlled such that a molar ratio of the diol thioether to hydrogen peroxide is 1:(2-10).

4. The continuous-flow preparation method of claim 3, wherein in step (S1), the first micro-channel reactor is controlled at 0-100° C.; and a residence time of the reaction mixture in the first micro-channel reactor is controlled to be 0.1-30 min.

5. The continuous-flow preparation method of claim 2, wherein in step (S2), a flow ratio of the reaction mixture to the water is controlled such that a molar ratio of the organic solvent to the water is 1:(0.5-5).

6. The continuous-flow preparation method of claim 5, wherein in step (S2), the second micro-channel reactor is controlled at −10-30° C.; and a residence time of the reaction mixture in the second micro-channel reactor is controlled to be 0.5-10 min.

7. The continuous-flow preparation method of claim 1, wherein the first micro-mixer and the second micro-mixer are independently selected from the group consisting of a static mixer, a T-type micro-mixer, a Y-type micro-mixer, a cross-type micro-mixer, a coaxial-flow micro-mixer, and a flow-focusing micro-mixer.

8. The continuous-flow preparation method of claim 1, wherein the first micro-channel reactor and the second micro-channel reactor are independently a tubular micro-channel reactor, or a plate-type micro-channel reactor.

9. The continuous-flow preparation method of claim 8, wherein an inner diameter of the tubular micro-channel reactor is 50 μm-10 mm; the plate-type micro-channel reactor comprises a first heat exchange layer, a reaction layer, and a second heat exchange layer successively arranged from top to bottom; the reaction layer is provided with a reaction fluid channel; and a hydraulic diameter of the reaction fluid channel is 50 μm-10 mm.
A continuous-flow preparation method of diol sulfone using a two-stage micro-reaction system. The system includes a first micro-mixer, a first micro-channel reactor, a second micro-mixer, and a second micro-channel reactor communicated in sequence. The method includes: feeding hydrogen peroxide, a catalyst and a diol thioether solution simultaneously to the first micro-mixer followed by mixing; feeding the reaction mixture to the first micro-channel reactor for continuous oxidation; and feeding the reaction mixture and water simultaneously to the second micro-mixer and the second micro-channel reactor for continuous quenching and crystallization to obtain diol sulfone.
